(19) United States
(12) Patent Application Publication
(10) Pub. No.: US 2002/0010502 A1
(43) Pub. Date: Jan. 24, 2002

(54) Thermotherapy method

(76) Inventor: John R. Trachtenberg, Toronto (CA)

Correspondence Address: Kohn & Associates, Suite 410, 30500 Northwestern Hwy., Farmington Hills, MI, 48334, US

(21) Appl. No.: 09/826,595

(22) Filed: Apr. 5, 2001

Related U.S. Application Data
(63) Continuation of application No. 09/190,786, filed on Nov. 12, 1998, now abandoned.
Application No. 09/190,786 is a continuation-in-part of application No. 09/053,477, filed on Apr. 1, 1998, now Pat. No. 6,064,914.
(60) Provisional application No. 60/076,619, filed on Mar. 3, 1998.

(51) Int. Cl. A61N 5/02; A61F 2/00

(52) U.S. Cl. 607/102; 606/105; 606/113

(57) ABSTRACT

A hydrodissection apparatus for treatment of the prostate of a patient using a moving apparatus for moving the prostate away from the adjacent rectum and heat used to heat the prostate while keeping the rectum protected from any damage that could be caused by the heat.

### CROSSREFERENCE TO RELATED APPLICATIONS

This is a continuation in part of Ser. No. 09/053,477 which is a conversion of provisional application No. 60/076,619.

### BACKGROUND OF THE INVENTION

1. Field of the Invention

The present invention relates generally to an apparatus and method for performing a thermal therapy patient treatment protocol. More particularly, the invention relates to a novel apparatus and method for physically separating organs to enable aggressive thermal therapy to be administered safely and relatively comfortably, on an outpatient basis, if desired.

Thermal therapy has been proven to be an effective method of treating various human tissues. Thermal therapy includes tissue freezing, thermotherapy, hyperthermia treatment and various cooling treatments. Thermotherapy treatment is a relatively new method of treating cancerous, diseased and/or undesirably enlarged human prostate tissues. Hyperthermia treatment is well known in the art, involving the maintaining of a temperature between 41.5 degrees Celsius through 45 degrees Celsius. Thermotherapy, on the other hand, usually requires energy application to achieve a temperature above 45 degrees Celsius for the purposes of coagulating the target tissue. Tissue coagulation beneficially changes the density of the tissue. As the tissue shrinks, forms scars and is reabsorbed, the impingement of the enlarged tissues, such as an abnormal prostate, is substantially lessened. Further, tissue coagulation and its beneficial effects are useful for treating cancerous tissue, because cancer cells are particularly susceptible to abnormal temperatures. Cancer cells can be treated in accordance with the present invention with temperatures in excess of 100 degrees Celsius without damage to the therapy applicator or discomfort to the patient.

The higher temperatures required by thermotherapy require delivery of larger amounts of energy to the target prostate tissues. At the same time, it is important to protect nontarget tissues from the high thermotherapy temperatures used in the treatment. Providing safe and effective thermal therapy, therefore, requires devices and methods which have further capabilities compared to those which are suitable for hyperthermia.

Although devices and methods for treating prostate cancer and benign prostatic hyperplasia have evolved dramatically in recent years, significant improvements have not occurred and such progress is badly needed. As recently as 1983, medical textbooks recommended surgery for removing cancerous or impinging prostatic tissues and four different surgical techniques were utilized. Suprapubic prostatectomy was a recommended method of removing the prostate tissue through an abdominal wound. Significant blood loss and the concomitant hazards of any major surgical procedure were possible with this approach.

Perineal prostatectomy was an alternatively recommended surgical procedure which involved gland removal through a relatively large incision in the perineum. Infection, incontinence, impotence or rectal injury were more likely with this method than with alternative surgical procedures.

Transurethral resection of the prostate gland has been another recommended method of treating benign prostatic hyperplasia. This method required inserting a rigid tube into the urethra 15. A loop of wire connected with electrical current was rotated in the tube to remove shavings of the prostate at the bladder orifice. In this way, no incision was needed. However, strictures were more frequent and repeat operations were sometimes necessary.

The other recommended surgical technique for treatment of benign prostatic hyperplasia was retropubic prostatectomy. This required a lower abdominal incision through which the prostate gland was removed. Blood loss was more easily controlled with this method, but inflammation of the pubic bone was more likely.

With the above surgical techniques, the medical textbooks noted the vascularity of the hyperplastic prostate gland and the corresponding dangers of substantial blood loss and shock. Careful medical attention was necessary following these medical procedures.

The problems previously described led medical researchers to develop alternative methods for treating prostate cancer and benign prostatic hyperplasia. Researchers began to incorporate heat sources in Foley catheters after discovering that enlarged mammalian tissues responded favorably to increased temperatures. Examples of devices directed to treatment of prostate tissue include U.S. Pat. No. 4,662,383 (Harada), U.S. Pat. No. 4,967,765 (Turner), U.S. Pat. No. 4,662,383 (Sogawa) and German Patent No. DE 2407559 C3 (Dreyer). Though these references disclosed structures which embodied improvements over the surgical techniques, significant problems still remain unsolved.

Recent research has indicated that cancerous and/or enlarged prostate glands are most effectively treated with higher temperatures than previously thought. Complete utilization of this discovery has been tempered by difficulties in protecting rectal wall tissues from thermally induced damage. While shielding has been addressed in some hyperthermia prior art devices, the higher energy field intensities associated with thermotherapy necessitate devices and methods having further capabilities beyond those suitable for hyperthermia. For example, the microwave-based devices disclosed in the above-referenced patents have generally produced relatively uniform cylindrical energy fields. Even at the lower energy field intensities encountered in hyperthermia treatment, unacceptably high rectal wall temperatures have limited treatment periods and effectiveness.

The prostate lies immediately above the rectum. The two structures are separated only by a thin fascial plane called the Denonvillier's fascia. This is composed of two layers which are in close contact. To kill prostate cancer cells within the prostate, the entire prostate, including the peripheral zone, must be included in the thermal window. However, because the rectum lies in intimate contact with the prostate, if one were to direct enough noxious agents, in most methods heat, to the periphery of the prostate sufficient to kill the cancer cells, one risks additionally damaging the adjacent rectum. This is the problem that the previously known methods have, which leads either to failure of treatment or morbidity.

In addition, efficient and selective cooling (for heat-based treatments) or warming (for freezing treatments) of the devices is rarely provided. This substantially increases patient discomfort and increases the likelihood of healthy tissue damage during benign prostatic hyperplasia treatments. These problems have necessitated complex and expensive temperature monitoring systems along the urethral wall. Satisfactory ablative prostate cancer therapy using extremely high or low temperature treatments cannot be undertaken without effective thermal control of the therapy device including effective cooling of exterior portions of the therapy device.

It would therefore be useful to utilize a method of treatment which enables the physician to both protect the adjacent rectum while still enabling the physician to direct enough heat to sufficiently kill the cancer cells.

### SUMMARY OF THE INVENTION

According to the present invention, a hydrodissection apparatus is utilized for treating the prostate of a patient by moving the prostate away from the rectum and then applying sufficient heat to the prostate to kill the cancer cells while protecting the rectum. Also included in the present application is a method of treating the prostate of a patient using the apparatus. Further included is a method of providing thermal therapy to prostate tissue of a patient by providing a fluid flow which thereby causes a physical separation of the prostate from the rectum.

### DESCRIPTION OF THE DRAWINGS

Other advantages of the present invention will be readily appreciated as the same becomes better understood by reference to the following detailed description when considered in connection with the accompanying drawings wherein:

FIG. 1 illustrates a front view of a human prostate and rectum in accordance with conventional medical knowledge;

FIG. 2 shows a front view of the prostate and rectum of FIG. 1 physically separated by a fluid;

FIG. 3 illustrates a side view of a prostate and rectum physically separated by a fluid;

FIG. 4 shows a front view of the prostate and rectum of FIG. 2 showing a device for providing the fluid and a fluid temperature sensor;

FIG. 5 shows a front view of a delivery system constructed in accordance with one form of the invention.

FIGS. 6(a) and (b) show (a) a transverse view of the configuration of the equipment used for hydrodissection; and (b) a sagittal view of the configuration of the equipment used for hydrodissection.

FIG. 7 shows a schematic saggital view of the equipment used for cooling and temperature monitoring in the hydrodissection space.

### DETAILED DESCRIPTION OF THE INVENTION

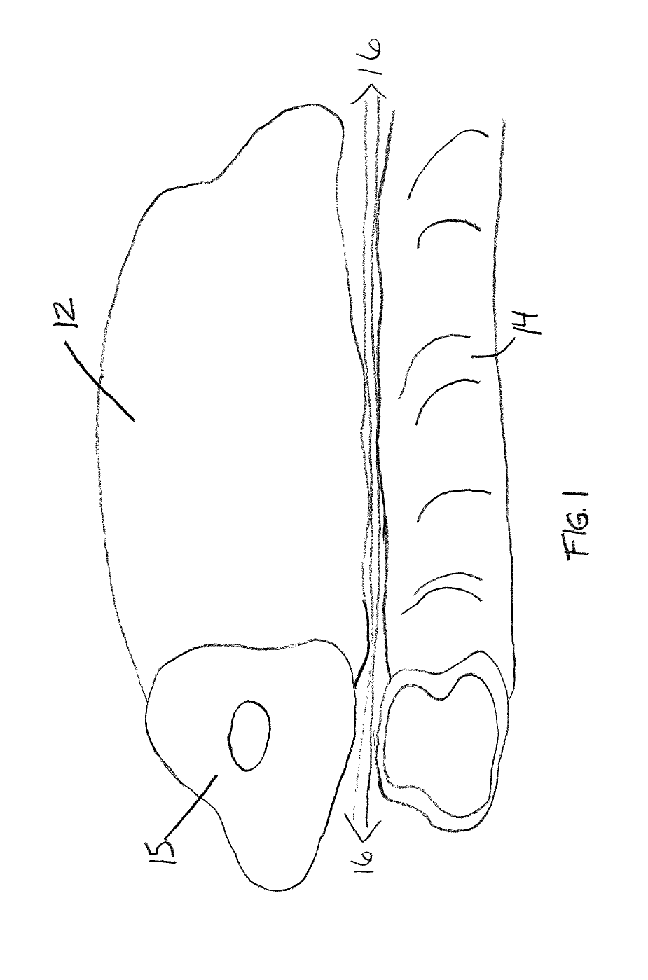

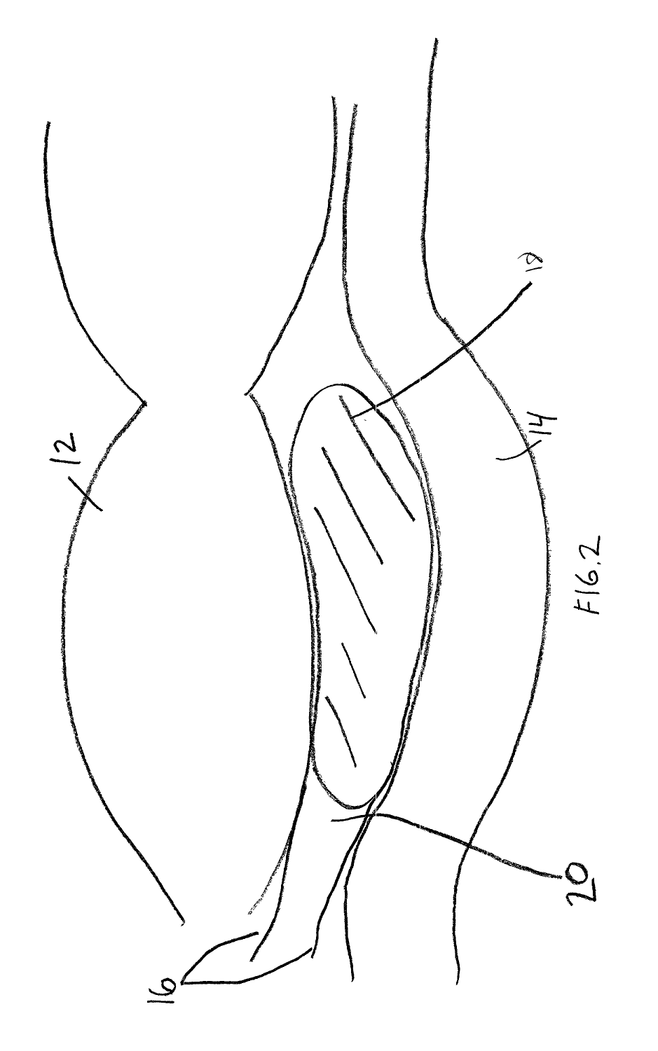

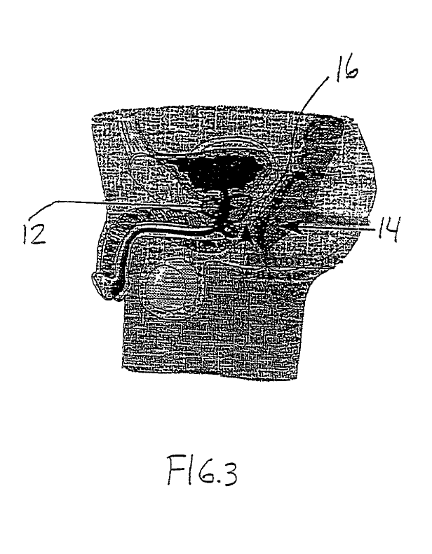

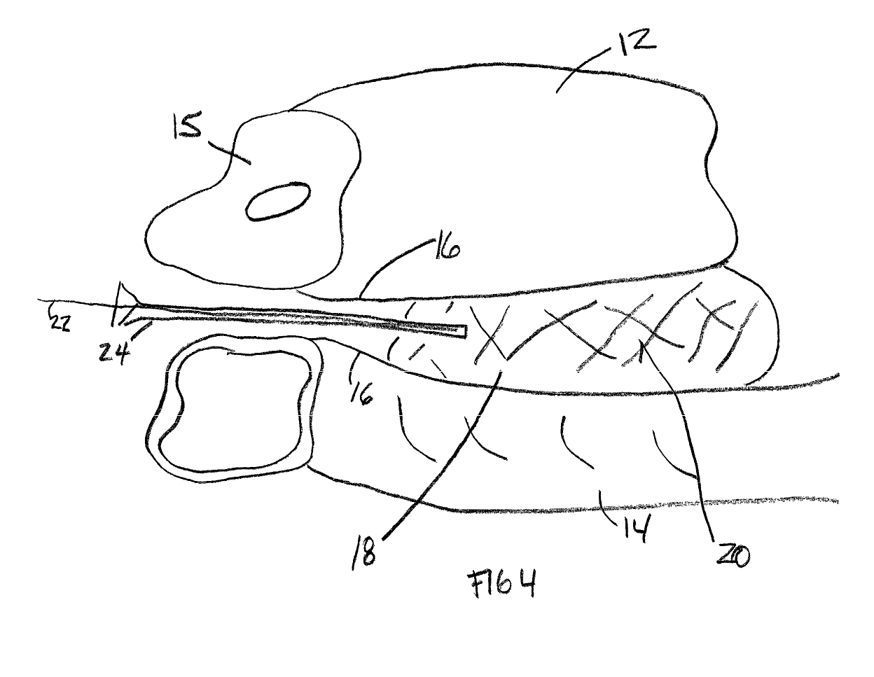

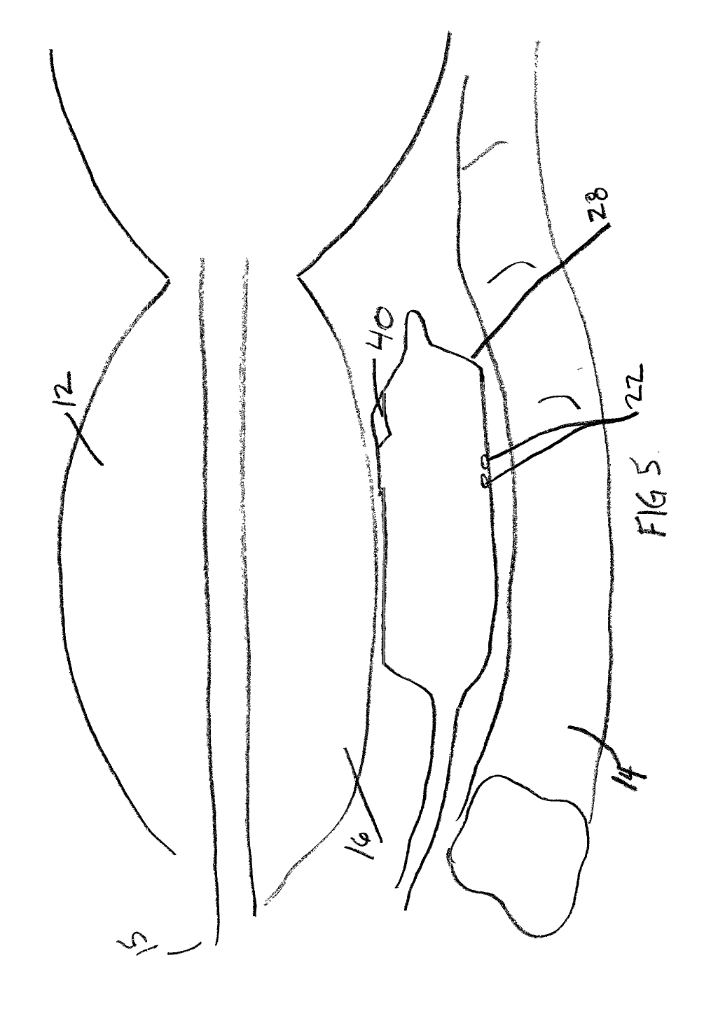

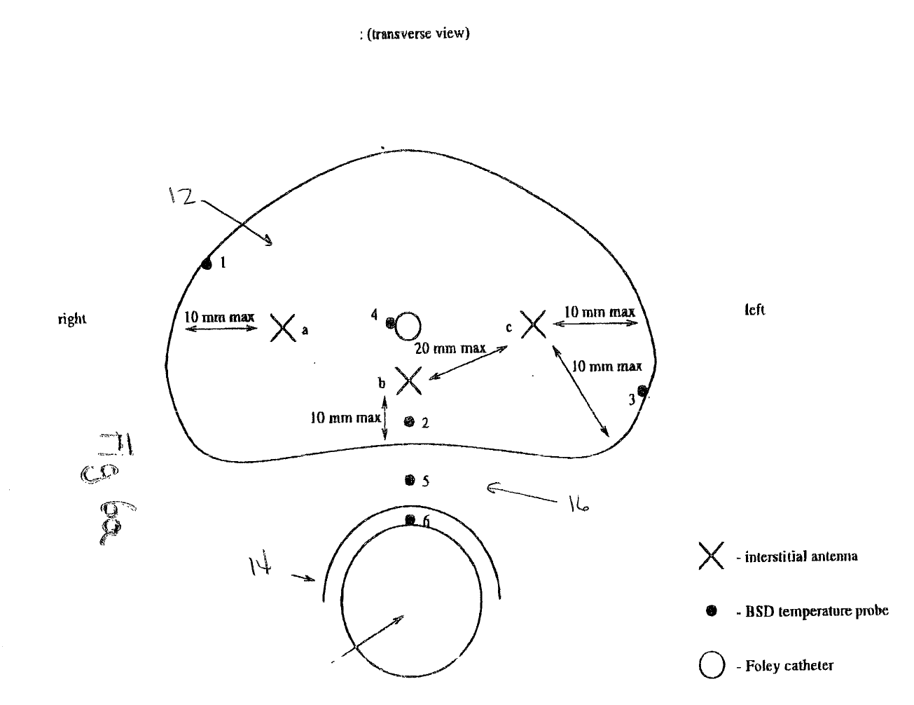

FIG. 1 illustrates a front view of a human prostate 12 located immediately above a human rectum 14 in accordance with well-known anatomical observations. The prostate and the rectum 14 are separated by a thin fascial plane called “Denonviller's fascia” or a “biplane fascial layer” 16. Denonviller's fascia is composed of two layers of fibrous membrane tissue in close contact. To kill prostatic cancer cells within the prostate 12, the entire prostate 12 must typically be subjected to the thermal therapy, regardless of whether heating or cooling techniques are utilized. Because the rectum 14 naturally lies in intimate contact with the prostate 12 and the biplane fascial layer 16, if one subjects the periphery of the prostate 12 to intense thermal therapy to kill all living tissue within, one risks damaging the portions of the rectum 14 close to the prostate 12. Such damage can lead to severe complications such as urethral or vasicle-rectal fistulae.

The present invention can use ultrasound or magnetic resonance or other imaging modalities to direct the percutaneous (through trans-perineal techniques or others) instillation of fluid flow 18 under pressure into the biplane fascial layer 16 (Denonvillier's fascia) to create a real space 20 from the pre-existing virtual space, thereby physically separating the rectum 14 from the prostate 12. Extremely low fluid pressures (i.e., gravity-fed flows) can be used in accordance with the invention if desired. The fluid flow 18 tracks into this fascial plane, physically and thermally isolating the rectum 14 from the prostate 12, and isolating the prostate 12 from lateral and inferior lying structures (e.g., the perineal diaphragm, sphincteric mechanism and neurovascular bundles). Fluid flow 18 can be continuously instilled to cool (or warm, as desired) and separate this space 20 and protect adjacent structures. Thermometry probes can be placed into the periphery of the prostate to ensure adequate temperatures to ablate cancer cells while temperature sensors 22 and pressure monitors in the fluid space can dictate the amount of fluid flow necessary to adequately protect adjacent structures. Additionally, mapping temperature probes are inserted into the prostate thermometry catheters. These mapping probes provide temperature data of the interstitial space between the prostate and the rectum along the length of the prostate throughout the treatment. Conventional intermittent trans-rectal ultrasound can also help ensure adequate continuing separation of vital tissues by the instilled cooling fluid flow 18.

In accordance with one preferred embodiment of the invention, a needle 24 is inserted at a location near or between the prostate 12 and rectum 14 to infuse a fluid flow 18 for cleaving or providing a space 20 physically separating the prostate 12 and rectum 14. It will be apparent that all of the organ separation methods described herein can be practiced from a variety of entry ports: transperineally, transrectally, transurethrally, suprapubically and others. The fluid flow 18 can be a cooling . . . solution (ionic or nonionic), an insulating medium (as in energy absorption), an energy reflecting medium for use with some trans-urethral therapy applications, a warming solution, air or a gas, or some type of gel. Infusing these types of agents essentially provides a space 20 to either help insulate the rectum 14 from the therapy or can provide a means to either augment the therapy or to provide the actual therapy itself.

The fluid flow 18 can be bolused in or continuously infused to provide proper maintenance of the space 20 between the organs and proper temperature of the fluid flow 18. The fluid flow 18 can also be recirculated into and out of the space 20 by the use of a multilumen catheter or by use of multiple catheters. For heat treatments, the fluid flow 18 can be cooled to provide cooling to the rectum 14. Alternatively, the fluid flow 18 can be maintained at a minimally therapeutic temperature by monitoring the temperature via a machine. The temperature data is collected to ensure that the cooling systems are effectively cooling the urethra and rectum. Therefore, monitoring of the fluid flow 18 temperature within the space 20 or in the delivered and returned solution temperature can be used to guide or enhance the treatment effectiveness. For cooling or freezing treatments of the prostate 12, the fluid flow 18 can be armed to ensure that the rectum 14 is provided a safety cushion such that the therapy inside the prostate 12 can be as aggressive as possible.

This space 20, once created, can also be used to provide a window within which to now deliver therapy, feedback regarding the extent of the treatment by providing more localized control or for various types of imaging (e.g., ultrasound). Further details for implementing those functionalities are described hereinbelow. This technique can be especially useful for prostate cancer which develops predominantly in the posterior and lateral edges of the prostate 12. The close proximity of the thermally sensitive rectum 14 to those commonly afflicted areas of the prostate 12 limits the effectiveness of conventional treatments. By utilizing the space 20 or window to now provide a means for directly treating these regions of the prostate 12 in a directional way, the rectum 14 can be protected from thermal damage, and the location of the cancer can be extremely aggressively treated in a safe and relatively comfortable manner. Therapy elements (energy sources) capable of providing desirably asymmetric energy patterns include, without limitation, laser, microwave (especially with some type of shielding (e.g., air) to avoid heating the rectum 14), cryosurgery, ultrasound (focused or diffuse) and diagnostic ultrasound. The diagnostic ultrasound and the therapeutic ultrasound can be combined into the same probe if desired.

The therapeutic element 36 can be directional, shielded or simply conventional. The element 36 can then be used to effectively treat the outer portions of the prostate 12. This approach can be used in conjunction with another form of treatment, either drug or device, and can be used with interstitial or intraluminal treatments. If needed, a conventional endoscope or similar device can be inserted to guide the application of the treatment under direct visualization.

The therapeutic element 36 can incorporate a locating means 40 whereby the location of the treatment can be confirmed, adjusted or maintained throughout the treatment. This locating means 40 can include, without limitation, a helium neon laser pointer for direct-vision or a mechanical/ultrasound opaque (i.e., metal) indicator on the probe itself. It can also comprise an ultrasound imaging device capable of monitoring the therapeutic effect in the tissue itself.

Additionally, the therapeutic effect is determined by monitoring the expected thermal damage volume of the prostate. This is calculated based upon the treatment temperatures as measured. This is achieved by digitizing the actual locations of the antennas and temperature probe catheters from the ultrasound scans obtained during the procedure. The positions and heating patterns of the antennas are then measured in muscle equivalent phantoms in pre-clinical testing, such that the expected temperatures during treatment are calculated based on the actual power delivered during treatment.

While prostate treatment uses of the present invention are described herein for illustrative purposes, it will be readily apparent that the present invention can also be used to treat other anatomical structures including, without limitation, structures inherent or attached to the rectum 14 itself (e.g., treating the wall of the rectum 14 or tumors associated with the rectum 14).

Thermal therapy delivery systems 50 can also be used as mechanical separators 28. The delivery system 50 can take a number of forms, such as the one described in co-pending U.S. patent application Ser. No. 07/976,232, the Detailed Description of Preferred Embodiments which is incorporated herein in its entirety. The delivery system 50 can include the ability to provide degassed and temperature regulated water flow into the delivery system 50 adjacent tissue to be treated. An example of such a suitable delivery system 50 is a single or multiple lumen device which circulates fluid, gas, gel and the like under pressure within a closed environment. The delivery system 50 is intended to be inserted into body cavities or interstitially. The delivery system 50 can be inserted into the body (organ) targeting a specific treatment site. The delivery system 50 can house a therapeutic element 36 such as laser, microwave, therapeutic or diagnostic ultrasound or simply a temperature sensor 22. The fluid flow 18 or infused agent can be recirculated under pressure or can remain static. This form of the invention can deliver therapeutic energy to internal body structures through a minimally invasive procedure.

The delivery system 50 is preferably small in diameter, being 9 French and under. Delivery systems 50 as small as 6 French have been used satisfactorily and are being further miniaturized. The delivery system 50 incorporates 360 degree radial cooling (or warming) which is essential for this intensive thermal therapy, especially for interstitial therapy, because it greatly reduces the potential for exit wounds which could result from both thermal or freezing technologies.

The delivery system 50 can be made out of extremely thin polymers, such as PET, which permits the use of very thin wall thicknesses, thereby minimizing the overall device size. This type of material is essentially nondistensible and can withstand high pressures without failure. This permits passage of fluid flow 18 or other media under pressure to provide flow without compromise of the structure. The delivery system 50 can also be made from typical catheter material with the size increasing due to the need for larger wall thicknesses.

The delivery system 50 can have a rigid structure that aids in insertion or could be made so thin that it essentially has no rigidity. The latter design can be inflated to provide the handling and insertion stability required. This has the advantage of permitting extremely thin wall thicknesses to be used, thereby maximizing throughput flow and/or minimizing overall size. The rigidity of the delivery system 50 can also be used in conjunction with a conventional sharpened tip at one end of the delivery system 50. The sharpened tip enables interstitial insertion of the delivery system 50.

The circulating fluid flow 18 could be either a cooling agent or a warming agent, whichever is required for the particular thermal therapy being utilized. For example, microwave therapy benefits from a cooled device whereby the cooling of the antenna provides a substantial increase in efficiency. The delivery system 50 preferably incorporates the therapeutic elements 36 with complete cooling or warming (via submersion) along the therapeutic element's 36 entire length. This configuration is the most efficient use of space, thereby resulting in a smaller profile.

The outer structure (lumen) 52 of the delivery system 50 can be made either nondistensible or moderately to fully distensible. A distensible outer lumen diameter can be changed even during a treatment to maintain desired contact with the surrounding tissue. This is important for therapies that benefit from intimate contact between the applicator and the tissue for efficient transmission of energy such as microwave, laser, ultrasound and the like.

The change in lumen 52 diameter can be accomplished via an active increase in the internal pressure of the delivery system 50. The pressure can be increased (inflated), decreased or otherwise controlled automatically (or manually) and triggered via the recording of reflected or lost power transmission which can be monitored real time. A conventional pump 60 or other inflation system can be controlled electronically for this purpose. This can be a feedback circuit to improve the efficient transmission of energy throughout the duration of the treatment. In this way, intimate contact between the delivery system 50 and the surrounding tissue can be maintained throughout the treatment, increasing the efficiency of the energy transmission.

Pressurization can also be a useful feature of the delivery system 50 for: clearing the pathway of air or impurities; cooling or warming; and reducing or eliminating modifications in the environment resulting from the treatment. For example, in microwave treatments, the cooling medium is typically a deionized solution such as distilled water. With the application of microwave energy, the microbubbles are produced along the antenna resulting in an increase in reflected power. This can develop into an almost total stoppage of emitted energy into the tissue. Pressurization desirably changes the degassing characteristics of the medium and can minimize the effect of microbubbles out of the energy emitting pathway. Air will block the transmission of most energy sources such as microwave and ultrasound. Laser will also see this as another interface which can result in overheating of the delivery system 50 in that region, possibly resulting in delivery system 50 or laser malfunction. Pressurization can therefore reduce or eliminate reflected power and can be varied throughout a treatment to compensate for changes in the reflected power levels that may occur.

Reflected power will also change according to the matching/mismatching characteristics of the environment surrounding the delivery system 50. This is especially true for microwave energy. Therefore, the measurement of reflected power can be used to correlate with tissue changes in the surrounding tissue. This measurement can, therefore, be used as a feedback mechanism for the progression of a treatment or for a regulating mechanism during a treatment. It can be used as a surrogate measure of tissue temperature or tissue destruction, and can also be used to determine if the treatment is being applied too aggressively. For example, if the therapy is too aggressive, the interface between the delivery system 50 and the surrounding tissue may change (e.g., dehydrate) which will impact the matching between the two entities. The severity of the mismatch will be reflected in an increase in the reflected power. This mismatch clinically results in a less effective administered treatment. By reacting to the change in the reflected power, the aggressiveness of the treatment can be modified to manage this event. Reflected power will change with changes in the temperature of the environment surrounding the delivery system 50. Accordingly, this measure can be used to estimate the temperature of the environment. This is the same for actual physical changes in the surrounding environment (e.g., denaturization, carbonization, dehydration, etc.); therefore, this measure can also estimate effects of a treatment upon the surrounding environment.

While preferred embodiments have been illustrated and described, it should be understood that changes and modifications can be made therein without departing from the invention in its broader aspects. Various features of the invention are defined in the following claims.

The above discussion provides a factual basis for the use of a method of providing thermal therapy to the prostate tissue of a patient. The methods used with and the utility of the present invention can be shown by the following non-limiting examples and accompanying figures.

### EXAMPLES

General Methods:

### Example 1

Treatment

The patient is administered prophylactic antibiotics on call to the operating room. In the operating room, the patient is placed on the cystoscopy table and a general anesthetic is administered. The suprapubic area and the perineum of the patient is then prepped and draped in the dorsal lithotomy position. The scrotum of the patient is secured to the anterior abdominal wall. The bladder is drained and a 16 French Foley catheter is then placed in the urethra 15. A transrectal ultrasound transducer is then placed in the rectum and the volume and configuration of the prostate 12 is confirmed. The Foley catheter is visualized in the urethra 15 in the sagittal plane.

The position and number of interstitial microwave antenna assemblies (MAA) to be inserted is based upon the volume and configuration of the prostate 12 which will be determined and planned using pretreatment transrectal ultrasound. The actual number of interstitial microwave assemblies (MAA) used will be determined based upon the volume and shape of the gland, as specified below:

| | |
|---|---|
| 45 to 75 cc gland | 4 MAA |
| 35 to 45 cc gland | 3 MAA |
| 25 to 35 cc gland | 2 MAA |

The treatment zone locations and number will be determined as to yield complete therapeutic heating of the prostate 12. The sites are plotted on a treatment map during pretreatment planning prior to insertion to achieve efficient isothermic heating of the tissue.

Placement of the intra-prostatic MAA is preceded by repeat topical antibacterial preparation of the perineum. In order to place the MAA, a needle and sheath assembly is first inserted, this assembly is a peel-away assembly. The needle and sheath assembly will be placed transperineally into the left lateral lobe of the prostate 12. The needle will be advanced along an axis 1.5 centimeters away from (as close as medically feasible) and roughly parallel to the prostatic capsule, adjacent to the bladder neck. The position is confirmed using transrectal ultrasound and the needle is repositioned as necessary. The MAA will be inserted into the lumen of the peel-away needle and sheath assembly and advanced until the distal tip reaches the end of the sheath. Proper MAA placement is confirmed when the MAA reaches the sheath hub. The peel-away sheath is then removed, leaving the MAA in place. This procedure is repeated until the predetermined MAA therapy plan has been accomplished. Using a similar technique, a 2-sensor thermosensor array will be inserted at a three or nine o'clock position laterally inside the gland at the capsule. Another 2-sensor array will be placed at the five or seven o'clock position. A single thermosensor array will be placed at the posterior mid line (recto-prostate interface) margins of the prostate 12.

Hydrodissection

The recto-prostatic interface will be delineated by transrectal ultrasound. A needle and sheath assembly will be guided with transrectal ultrasound into this space. The rectum will be separated from the prostate 12 by hydrodissection within the recto-prostatic space. This will be accomplished by inserting a needle and sheath assembly into the recto-prostatic space and connecting a continuous saline solution drip infusion. The hydrodissection will be confirmed by transrectal ultrasound. A single array thermosensor will be inserted via a Y-connector through the needle and sheath assembly into this space. The temperature within the hydrodissection space will be continually monitored. The infusion flow rate will be adjusted to maintain a maximum temperature of 43.5 degrees Celsius within the space. If the monitored temperature rises above 45 degrees Celsius, the flow rate will be increased. If the increased flow rate does not decrease the temperature below 45 degrees, the power of the respective treatment zone(s) will be turned down as described below. A rectal probe with two thermosensors spaced two centimeters apart in length will be placed in the patient's rectum. A transrectal ultrasound transducer with the same two thermosensor array may be used to monitor rectal temperatures and hydrodisection.

A urethral cooling assembly will be coated with sterile lubricant and inserted into the urethra 15 with the anchor balloon inside the urinary bladder. The anchor balloon will be inflated with 7 cc of sterile water and traction will be applied to ensure that the applicator is in the proper position. The proper position will be with the proximal side of the anchor balloon seated against the urinary bladder neck. Either a rectal probe or a transrectal ultrasound transducer will be inserted into the rectum 14. The rectal probe or transrectal ultrasound transducer will have a two sensor thermosensor array spaced by two centimeters. It is preferred to use the transrectal ultrasound transducer/thermosensor array to visually monitor the hydrodissection space. During the procedure, MAAs, thermosensor fiber arrays, and water connections for the urethral and rectal cooling devices will be attached to the respective system connectors and the treatment program will be initiated.

Power Ramp-up Procedure

The microwave power will be initiated at 5 watts. The power will be manually increased in 5 watt increments every two minutes to a maximum of 25 watts.

| Time: | Wattage: |
|---|---|
| 0-2 minutes | 5 watts |
| 2-4 minutes | 10 watts |
| 4-6 minutes | 15 watts |
| 5-8 minutes | 20 watts |
| 8-10 minutes | 25 watts |

The power of the respective treatment zone will be turned down in 2.5 watt increments when the interstitial thermosensor reaches 75 degrees Celsius. The power will be lowered 2.5 watts every one minute until the interstitial temperatures within the respective treatment zone(s) are stabilized within the target treatment temperature range, 55 to 75 degrees Celsius.

The treatment time will start when all interstitial thermosensors have reached the treatment range, 55 to 75 degrees Celsius. A minimum temperature of 55 degrees Celsius must be attained to start the treatment clock. The treatment will be 15 minutes at temperatures within the treatment range. If the treatment range is not attained, the treatment will be 20 minutes in length plus the 10 minute power ramp-up time, or 30 minutes total.

The rectal temperature limit will be 43.5 degrees Celsius as measured on the surface of the cooled rectal probe or ultrasound transducer/thermosensor array. If the rectal temperature limit is exceeded, the power will be decreased as described above.

The target intraprostatic temperature during treatment is 75 degrees Celsius for 15 minutes within the target temperature zone to 0.5 centimeters of the margin of the prostate 12. A gradient of 75 to 45 degrees Celsius within this lateral heating zone of the prostate 12 has been calculated. The target temperature zone is a cylinder of tissue extending the length of the prostate 12, roughly parallel with and centered 0.2 centimeters lateral to the urethra 15, extending outward to the prostatic capsule. The urethral cooling assembly consists of a 9 French catheter which inflates to 18 French during treatment. This assembly constantly circulates 30 degrees Celsius cooled water to cool the urethral mucosa. Similarly, the rectal probe contains two thermosensors in a linear array designed to interrupt the treatment and shut down the microwave power if the rectal mucosa temperature exceeds 43.5 degrees Celsius. Additionally, the operator will be alerted via a “pop-up” dialogue box on the treatment screen prior to interruption of treatment. The rectal probe is cooling and protecting the rectal mucosa. Furthermore, the rectal mucosa has been separated by hydrodissection from direct contact with the heated prostate 12 and the created hydrodissection from direct contact with the heated prostate, and the created hydrodissection space is actively cooled via a saline infusion.

The location and size of the target temperature zone will allow for glandular asymmetry and normal anatomic variation in the angle and curvature of the urethra 15 through the prostate 12. The thermosensor's readings will be visually monitored throughout the therapy treatment. At the moment any of the intra-prostatic thermosensors reach 75 degrees Celsius, the microwave power will be lowered at 2.5 watt increments every one minute until the intra-prostatic temperatures are stabilized within the treatment range, 55 to 75 degrees Celsius. Due to the variable heat transfer rates in tissue, some overshot and lag response in temperature beyond the 75 degree Celsius limit is expected and the operator must take each patient's response characteristics into consideration when adjusting the microwave power levels. If the intra-prostatic temperature continues to fall after the microwave power has been decreased, the procedure described for lowering the power will be reversed to maintain the intraprostatic temperatures within the treatment range.

### Example 2

Materials and Methods

Thirteen patients who had failed external beam radiation therapy were treated with transperineal interstitial microwave therapy. All patients were noted to have had a rising PSA. Mean PSA at treatment was 17.8 ng/ml (range 0.2 to 120 ng/ml). All were subjected to prostatic biopsy and had histologic evidence of residual prostatic cancer. All patients were at least 18 months after therapy. No patient had received hormone therapy and all patients had a normal serum testosterone prior to treatment. All patients underwent a bone scan, a pelvic CT scan and an endo rectal magnetic resonance scan of the prostate. No patients had evidence of extraprostatic disease. All patients gave informed consent to be part of the experimental trial. Under general or epidural anesthetic with the patient in the lithotomy position, the suprapubic and perineal area was prepped. A 16 french Foley catheter was placed into the urethra and the scrotum was secured to the anterior abdominal wall. A 7-MHz transrectal ultrasound biplane probe was then placed in the rectum, and the volume and configuration of the prostate were confirmed. Guided by trans rectal ultrasound, four custom designed (six french) needles with a peel-away introducer set (Cook Canada) were placed transperineally in the prostate. These needles were placed along the center line of each quadrant of the transverse image of the prostate such that the apparent volume of microwave radiant energy (2 cm×3 cm elipse) would intersect and entirely fill the prostate. The right and left anterior, and right and left posterior needles had their peel away assembly removed and replaced with microwave antenna assemblies (Dornier Medical Systems, Inc. Kennesaw, Ga.). These helical antennas were individually cooled to minimize impedance mismatching with the tissue and they were powered at 915 MHz. A left lateral prostate and medial posterior recto-prostate interface needles were also placed and four channel fibreoptic thermosensors (four sensors, 1 cm apart) were inserted through their lumens. These probes were used to continuously monitor microwave generated temperature levels at the lateral and medial periphery of the prostate throughout the procedure (FIGS. 1A, B). In prostates whose volume could not accommodate four antenna assemblies two were placed in a similar fashion. Five patients were treated in a similar fashion, but were also monitored in a superconducting 1.5T General Electric body coil magnetic resonance scanner using phase shift thermometry to assess the prostate and pelvic structure temperatures online. Hydrodissection was accomplished by guiding another needle into the virtual space between the prostate and the rectum (FIG. 1). Saline was infused to separate the prostate from the rectum and genitourinary (GU) diaphragm. The process was observed by ultrasound in transverse and saggital views. The space was maintained by continuously infusing a sterile saline solution such that separation of the prostate from the rectum and prostate from the GU diaphragm was visualized on transrectal ultrasound. A fibre optic temperature sensor was placed through the infusion cannula into the area of hydrodissection to monitor recto-prostate interface temperatures. The rate of infusion of the saline solution could be adjusted to maintain a predetermined safe temperature. The Foley catheter was removed. Urethral and rectal cooling assemblies (Dornier MedTech, Inc. Kennesaw, Ga.) were inserted and water circulated to further protest these tissues. Power was applied to the antenna assemblies at five watt increments every two minutes to a peak wattage of 40, or until the target temperature was reached. Urethral and rectal temperatures were also monitored to assure tissue temperatures did not exceed 45° C. and 43.5° C. respectively. The prostate was then heated to a plateau peripheral temperature of 65° C. for 15 minutes. At the end of the procedure all hardware was removed and the Foley catheter was replaced. The patients were monitored overnight and were discharged the following day. The Foley catheter was removed four to seven days later.

Patients were assessed at month 1, 3, 6 12, 18, and 24. At each visit a history to include adverse events and physical exam (including DRE) was performed. In addition, serum PSA was determined. At months 3, 6, and 12 a gadolinium enhanced endorectal magnetic resonance scans were performed. At months 6, 12, and 18 trans rectal ultrasound guided biopsies of the prostate were performed.

Magnetic Resonance (MR) Imaging

All MR imaging was performed on a superconducting

1.5T unit (General Electric Medical Systems, Milwaukee, Wis.). A dual ring endorectal coil (Medrad Inc., Pittsburgh Pa.) and 1 mg of glucagon intravenously administered were used in all patients. T1-weighted images (TR 534, minimum TE) were obtained in the axial plane pre and post gadopentetate dimeglumine (Magnevist; Berlex; Montreal, Canada) administration (0.2 ml/kg). In addition, coronal and sagittal T1-weighted images (534, minimum TE) were also acquired after contrast administration. All enhanced T1-weighted images were obtained with chemical fat suppression. Pre-contrast fast spin echo (FSE) T2-weighted fat suppressed axial and coronal images (4900, effective TE 140, ETL of 12) were also acquired. Two excitations were obtained for T1-weighted images and three for T2-weighted images. Slices were 4 mm thick with a 1 mm skip and a 12 cm field of view. The matrix size was 256×192 except for axial FSE T2-weighted images when a 256×224 matrix size was used.

Results

Thirteen patients were treated. Two patients had serum PSA greater than 10 ng/ml (48 and 120). The remaining 11 patients had a pretreatment PSA of <10 ng/ml. The mean pretreatment PSA of all patients was 15.6 ng/ml (group 1) and for the 11 patients whose PSA was <10(group 2) it was 7.0 ng/ml. In all patients there was a decrease in serum PSA. In group 2, mean PSA declined from 7.0 to 1.7 and 1.1 ng/ml at 3 and 6 months respectively (FIG. 1). Seven of these 11 patients had a PSA of less than 0.5 ng/ml at six months. All of these patients underwent transrectal ultrasound guided sextant biopsies. In all patients, no evidence of malignancy was noted.

Fibromuscular cells and rare glandular elements were noted. Of the remaining four patients, two were considered technical failures. In one patient, a microwave generator failure caused only one side of the prostate to be heated, and in another, the configuration of the prostate did not allow for insertion of the antennas in the optimal position. After an initial drop in PSA, the marker returned to the baseline. In the two remaining patients PSA declined initially, but climbed after that. Prostate biopsies in these later two patients were negative, suggesting adequate local tissue destruction. The two patients treated with initial Serum PSA >10 showed interesting characteristics. In one patient whose initial PSA was 48, there was an initial, very gratifying decline of PSA to undetectable levels at months one and two. However, at months three and six, PSA was 0.2 and 1.4 ng/ml respectively, suggesting survival and recovery of some cancer tissue. At 12 months, this patient had a PSA of 8.1. The remaining patient had an initial PSA of 120. His PSA declined marginally to 110 ng/ml at one month, but climbed to 140 by three months. Surprisingly, his bone scan remains negative. All patients underwent pretreatment and three month gadolinium enhanced endorectal magnetic resonance scanning. Overall successful treatment decreased prostate volume by 27% and showed a marked devascularization of the prostate with preservation of tissue in the peri urethral area. These results form the basis of another publication.

Details of the magnetic resonance scanning suggest that areas that were incompletely ablated in the peripheral zone as seen in post treatment gadolinium enhanced MR scans may be the sites of tumor recurrence.

Two men became totally incontinent after treatment, and have remained so for at least six months. One of these patients developed a prostatic abscess that drained transurethrally. One further patient has noticeable stress incontinence. This patient was noted to have a large necrotic cavity that also drained transurethrally.

No patient had a rectal fistula. There were no cases that required rehospitalization.

Only one patient was potent prior to therapy. He remains sexually active, but claims “diminished” erectile function. This patient also complained of perineal discomfort that gradually subsided after two months.

Discussion

Various series, Yerushalmi et al. and Mendecki et al., have reported the use of hyperthermia in the treatment of prostate cancer. These treatment regimes have usually been limited to temperatures of less than 45° C. and have been used with other forms of therapy such as radiation, Anscher et al. Higher temperature regimens called thermotherapy or thermoablation have been designed to be used as mono therapy to destroy viable prostate cancer by high (>45° C.) temperatures alone. While heating the prostate to this temperature is feasible by a variety of techniques (laser, focused ultrasound, RF), the system reported above is unique by virtue of the combination of multi-antenna microwave heating of the prostate and protection of the rectum and sphincteric mechanism by a separation and cooling technique called hydrodissection, and the integration of on line magnetic resonance scanning to visualize the extent of heating in the entire prostate. These three factors have been utilized to improve upon prior attempts of thermoablation of the prostate. Each of these design elements remains in the development stage but has contributed to the clinical results. Individual controlled microwave antennas have allowed for simultaneous high power heating of the entire prostate. Since the vascularity of the prostate is markedly different in different zones, the ability to tailor rapid energy delivery to obliterate the blood supply in each zone based on the temperature in the zone is advantageous. This has resulted in a quick rise to effective heating temperature with the ability to heat different parts of the prostate at different rate. Hydrodissection has been the enabling element of the treatment. Although heating of the prostate is achievable by a variety of means, ablation of the peripheral zone of the prostate necessitates intense heating of not only the prostate, but of the adjacent tissues. Hydrodissection separates the prostate from surrounding tissues leaving a fluid filled space that acts as a heat sink that can, if necessary, be actively cooled. In addition, in our system, a thermosensor placed in this space allowed for measurement of this interface temperature. This added both a safety and function aspect by allowing active cooling if necessary. These preliminary results are in marked contrast to other contemporary attempts to ablate the prostate using thermal energy. Gelet describes the side effects that occurred in the preliminary use of trans rectal focus ultrasound to ablate prostate cancer, Gelet et al. (1996) and Gelet et al. (1998). In their first series of patients with primary prostate cancer, they reported a 50% incidence of serious side effects. These include recto-urethral fistulas, rectal burns, incontinence and bladder neck contracture. The cases described in the present report had a better side effect profile and were all in patients who had failed radiation therapy and would be more likely to be damaged by thermal injury.

Placement of the antenna assemblies, thermosensors, and hydrodissection cannula was done by conventional transrectal ultrasound technology which simplified the learning of the procedure. An additional, but optional procedure, used in five of our patients has been magnetic resonance imaging. This procedure has been used to verify placement of the antennas, devascularization of the gland at the end of the procedure with gadolinium enhanced images, and online measurement of tissue temperature. This last technique has added a further degree of precision to the procedure. “Cold” or poorly heated spots anywhere in the prostate are immediately visible. This imaging process allows for more precise heating of the prostate. It also adds a further degree of safety by visualizing where heating is adequate and where it is not or should not be occurring (e.g. the rectum).

Nonetheless, these results suggest that the prostate can be heated to high temperatures safely and that large amounts of prostate tissue can be ablated. The early clinical results in terms of negative biopsies and low serum PSA is gratifying, but remains too early in the course of follow up of the treatment to allow for definitive conclusions as to the effectiveness of this therapy. What is remarkable is the low rate of side effects in this group of high risk patients. The results of this trial suggests that this mode of therapy is safe and deserves further study. It also raises the question of whether selected patients might benefit from this treatment as primary treatment for prostate cancer.

### Example 3

Objectives

To establish the safety of percutaneous microwave thermal therapy in patients with recurrent prostate cancer. Also, to demonstrate that both the urethra and the rectum are protected during treatment using interstitial temperature measurements. Further, to demonstrate that the posterior and lateral margins of the prostate reach cytotoxic temperatures.

To provide preliminary data as to the outcome of percutaneous thermotherapy in patients with local recurrence of prostatic carcinoma following definitive radiotherapy as measured by:

Time to treatment failure.

Time to disease progression.

Quality of life.

Study Design

This is a pilot study designed to include 25 evaluable patients who have histologically proved recurrent or persistent adenocarcinoma of the prostate following definitive radiotherapy applied either externally, using brachytherapy techniques, or combination external and interstitial radiotherapy. This number of patients provides a 0-13.72% confidence interval (based on a p value of 0.05) on the probability that the procedure results in no major side effects based on the assumption that no major side effects are observed in these 25 patients. The study is terminated if any major side effects caused by the treatment are observed. The possible side effect of most concern is a rectal fistula.

Selection of Patients

Inclusion Criteria:

All of the following criteria must be satisfied:

Patients must have histologic proof of adenocarcinoma of the prostate 12 months or longer following definitive radiotherapy (external brachytherapy; or combination external and interstitial radiation).

Patients must have disease confined to the prostate and or local area (Stage A, B, or C disease) without evidence of regional and or distant disease.

Patients must have recent (within 2 months) negative bone scan and negative CT scan of the abdomen and pelvis.

Patients must have prostatic volume <50 gm as determined by calculated volume using transrectal prostatic ultrasound.

Patients must have serum prostatic specific antigen (PSA) equal to or less than 50 ng/ml using Hybritech or Abbott assay.

Patients must have a life expectancy of at least 5 years.

Patients must sign an Informed Consent indicating that they are aware of the investigational nature of this study, in keeping with the policies of this hospital.

Exclusion Criteria:

Patients having one or more of these characteristics may not participate in this study:

Patients who have received previous or current hormonal treatment or chemotherapy for prostatic carcinoma.

Patients unable to tolerate transrectal ultrasound.

Patients with an elevated serum prostatic acid phosophatase determined by the enzymatic (Roy) method.

Patients with metalic implants in or close their pelvis, e.g. hip prosthesis.

Pretreatment Evaluation (See Appendix 6.1 Pretreatment measurements)

Complete history and physical examination to include performance status as recorded using Zubrod and Karnofsky scores (Appendix B), and Quality of Life Assessment (Appendix C).

Histologic confirmation by a pathologist of adenocarcinoma of the prostate persistent or recurrent following radiotherapy.

Laboratory studies to include complete blood count (CBC), PSA (Hybritech assay), prostatic acid phosphatase (enzymatic method), SMA, and urinalysis.

Radiologic studies to include within 2 months of thermotherapy: chest x-ray, bone scan, and abdominal and pelvic CT or MR scan.

Uroflowmetry (peak urine flow rate and postvoid residual).

Transrectal prostatic ultrasound with volume determination calculated based on length, width and height using formula L×W×H/2.

Assessment of local extent of disease using digital rectal examination (Appendix D).

Hydrodissection

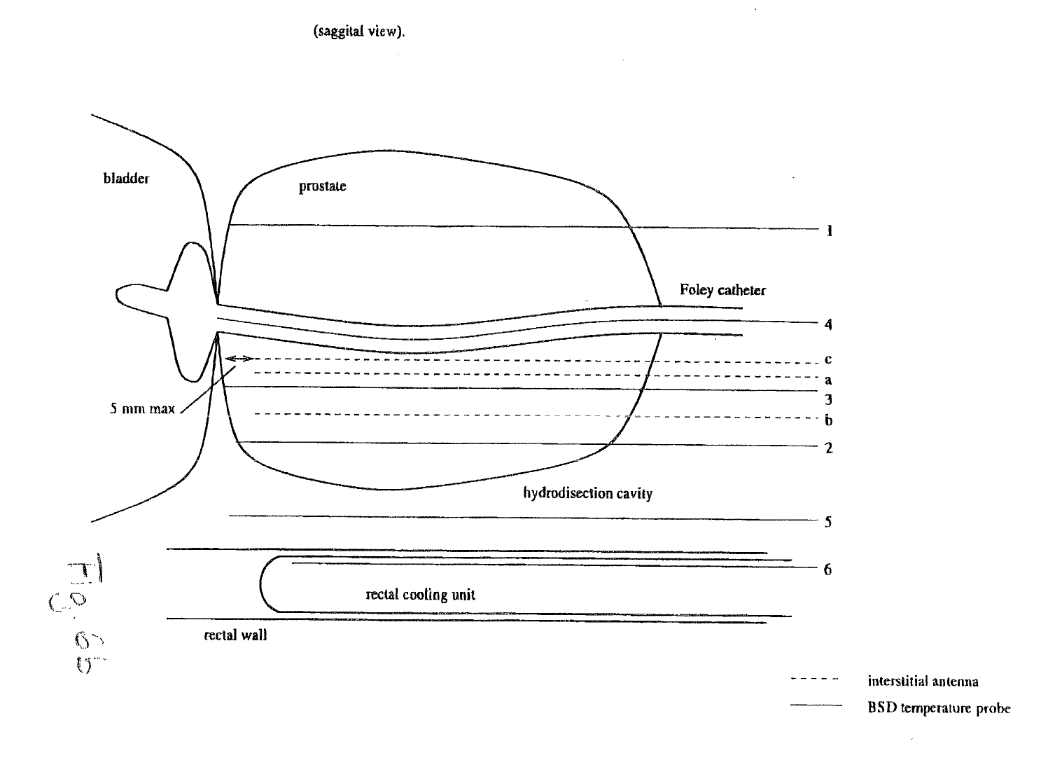

The microwave antennas, thermotherapy probes, and cooling mechanisms are inserted as shown in FIGS. 6 A and B. This probe placement is based on computational models, laboratory tests and animal experiments to determine the heating patterns of the microwave antennas.

The antennas and catheters for thermometry probes are inserted through peel away sheaths that are inserted into the tissue under transrectal ultrasound guidance. The antennas extend to a point 1 cm from the superior edge of the prostate. The catheters for the thermometry probes extend to the superior edge of the prostate (the thermometry probes map the entire length of the prostate). The Foley catheter is inserted into the urethra under transrectal ultrasound guidance and is secured at the distal end in the bladder by injecting fluid into the Foley balloon. Mapping temperature probes are inserted into the three interstitial prostate thermometry catheters and into the thermometry catheter attached to the Foley.

The hydrodissection tube is inserted into the tissue between the prostate and rectum under transrectal ultrasound guidance. Saline is injected until a space of at least 1 cm is created between the prostate and rectum. This space is maintained by attaching the tube to a saline drip. A thermometry catheter probe is inserted into the tube through a locking dam to a point posterior to the superior edge of the prostate. A mapping temperature probe is inserted into the catheter to provide temperature data in the interstitial space between prostate and rectum along the length of the prostate throughout the treatment.

At this point in the procedure, the transrectal ultrasound is removed and a cooled plastic insert is placed in the rectum with a sixth mapping temperature probe inserted into a thermometry catheter that is attached to the insert.

The BSD machine is turned on and temperature data collected to ensure that the cooling systems are effectively cooling the urethra and rectum. Power to the antennas a) and c) is then turned to 15 Watts each. The power is adjusted to achieve an approximately equal rate of temperature rise at each of the prostate tissue measurement points. A delay of a few minutes is observed before temperatures rise significantly at the prostate margin. Urethral and rectal temperatures should not be allowed to rise above 42 C. The interstitial space between rectum and prostate should also remain below 42 C.

The prostate tissue margins should be reached in excess of 55 C and be maintained above that temperature for 15 minutes. This will result in complete destruction of living tissue in the target area of the prostate. The thermal mapping will be used to determine the extent of damage along the length of the prostate.

After power is turned off, the temperature mapping continues until all tissue temperatures are below 42 C.

The antennas and cooling devices are removed and the patient revived.

Postoperative Assessment (See Appendix A):

Post-treatment measurement:

4 weeks:

1a digital rectal examination of the prostate;

1b urinalysis;

1c prostatic specific antigen;

1d uroflowmetry;

1e quality of life and performance questionnaire;

1f prostatic ultrasound with volume measurements.

8 weeks:

2a digital rectal examination of the prostate;

2b urinalysis;

2c prostatic specific antigen;

2d uroflowmetry;

2e quality of life questionnaire.

3 months:

3a digital rectal examination of the prostate;

3b urinalysis;

3c prostatic specific antigen;

3d uroflowmetry;

3e quality of life and performance questionnaire;

3f prostatic ultrasound with volume determination.

6 months:

4a same as for 3 month evaluation (see 3c)

4b selected site biopsies of the prostate under ultrasound guidance.

Every 3 months following 6 month evaluation:

5a digital rectal examination;

5b prostatic specific antigen;

5c prostatic ultrasound (for one year)

5d uroflowmetry (for one year)

5e quality of life and performance questionnaire.

Frequency of measurements:

Data will be collected pretreatment at 4 weeks, 8 weeks, 12 weeks, and every 3 months until local recurrence and/or progression of disease is documented. Direct assessment of adverse events will be specifically obtained at each visit.

Assessment of Results:

The first objective of this study is to determine the safety of thermotherapy for treatment of recurrent prostatic carcinoma following radiation therapy. All patients are to be followed at 3 months intervals with careful assessment for any adverse affects. Should major complications ensue, the study will be terminated.

The second objective of this study is to evaluate the efficacy of thermotherapy to eradicate malignant disease recurrent or persistent, following radiation therapy.

Careful evaluation of these initial 15 patients should provide preliminary answers to its efficacy within 6 months based upon biopsy results as well as the findings on serial PSA determinations and digital rectal examinations. Local failure will be confirmed by repeat prostatic biopsy. A rise in prostatic specific antigen by more than 50% will necessitate a repeat bone scan, acid phosphatase, and prostatic biopsy.

Complete objective response: No evidence of residual cancer as demonstrated by normalization of serum PSA (<4.0 ng/ml) and negative prostate biopsies.

Partial objective response: Reduction of serum PSA by at least 50% without normalization and no evidence of local tumor enlargement as assessed by digital rectal examination. Persistent tumor may be observed on biopsy. Patients with persistent disease within 6 months of treatment may undergo a repeat thermotherapy procedure. If residual cancer is detected at one year, then the patient is considered a treatment failure and taken off the study.

Treatment failure: Less than 50% reduction in serum PSA or absence of normalization of PSA, and or positive prostatic biopsy at one year following completion of treatment. Patients failing treatment are to be withdrawn from the study and offered alternative therapy.

Disease progression: Rise in serum PSA by more than 25%, enlargement of local tumor, or evidence of metastases on bone scan, CT or MRI. Patients with documented disease progression are taken off the study and may be offered alternative therapy.

In addition to the clinical measures described, one calculates the expected thermal damage volume in the prostate based on the treatment temperatures measured. This is achieved by digitizing the actual locations of the antennas and temperature probe catheters from the ultrasound scans obtained during the insertion procedure. Using the known positions and the hearing patterns of the antennas measured in muscle equivalent phantoms in the pre-clinial testing of this equipment, one calculates the expected temperatures during treatment based on the actual power delivered during treatment. Therefore, a three-dimensional temperature map is produced as a function of time during the entire treatment. The accuracy of the calculation is ensured by comparing the calculated temperatures with the measured temperatures at the locations of the temperature measurement probes.

The results of the simulated treatment are used to calculate the expected damage volume (volume where at least 99.9% cell death should be produced). This volume is correlated with pre-clinical assessments of tumor stage and outcome measures described above. This data provides information as to the importance of completely destroying the target volume during microwave thermotherapy.

Throughout this application, various publications are referenced by author and year. Full citations for the publications are listed below. The disclosures of these publications in their entireties are hereby incorporated by reference into this application in order to more fully describe the state of the art to which this invention pertains.

### Example 4

Microwave Thermotherapy for Patients with Recurrent or Persistent Localized Carinoma of the Prostate following Radiation Therapy (Update of Progress)

Milestones

New Implant Catheters to house the BSD temperature probes

New design allowing easy assembly of the hydrodisection space unit, and convenient access for saline coolant and the BSD temperature probe

New temperature probe catheters for the hydrodisection space and rectal cooling units

Continuous VHS recording of the ultrasound catheter guidance session

Still print of the ultrasound image of the hydrodisection space

Initial contacts to recruit more patients

Treatment Update

To date, four patients have been treated. The antenna and temperature probe arrangement shown in Figure GA was employed in the last three treatments (the first patient did not have antenna b placed). Antenna b is intended to be powered only if required, and in only the last patient was power actually supplied to this antenna.

In the last treatment, the temperature at probe 1 at the depth of the microwave radiators was above 60° C. for approximately 15 minutes, and was above 50° C. for approximately 28 minutes. The temperature at probe 2 at the same depth was above 60° C. for approximately 5 minutes, and was above 50° C. for approximately 15 minutes. At probe 3, the temperature was above 50° C. for approximately 5 minutes. These temperatures are cytotoxic, and therefore are expected to show promising results of follow-up tests. Temperatures in the hydrodisection space and rectum were well within safe limits.

Recent Efforts: Equipment Improvements

The has been conception of new designs for temperature probe catheters, a hydrodisection space cooling and temperature monitoring assembly, and antenna introducers. Most of these items were acquired, and in most cases then modified. These new designs are required to make ultrasound guided insertion of these items to mm precision less labor-intensive, and to reduce the total treatment time.

New Desiqns:

1. Implant Catheters for Temperature Probe Insertion (Supplied by Cook Canada Inc.):

These replace the previous “splittable needle” introducer sets which were used for temperature probes 1, 2, and 3 (FIG. 6A). The splittable needles are cumbersome to manipulate, require two people to place properly, and have very sharp edges that can easily cut a finger. The Implant Catheter consists of a closed-ended, pionted polyethylene catheter (6 French size, 15 cm length, to be replaced by a 20 cm model) Luer-locked to an insertion trocar. This two-piece set was originally designed for radioisotope delivery. After implantation of the set, the trocar is removed and the BSD temperature probe conduit is Luer-locked to the cathter.

2. Hydrodisection Space Improvements:

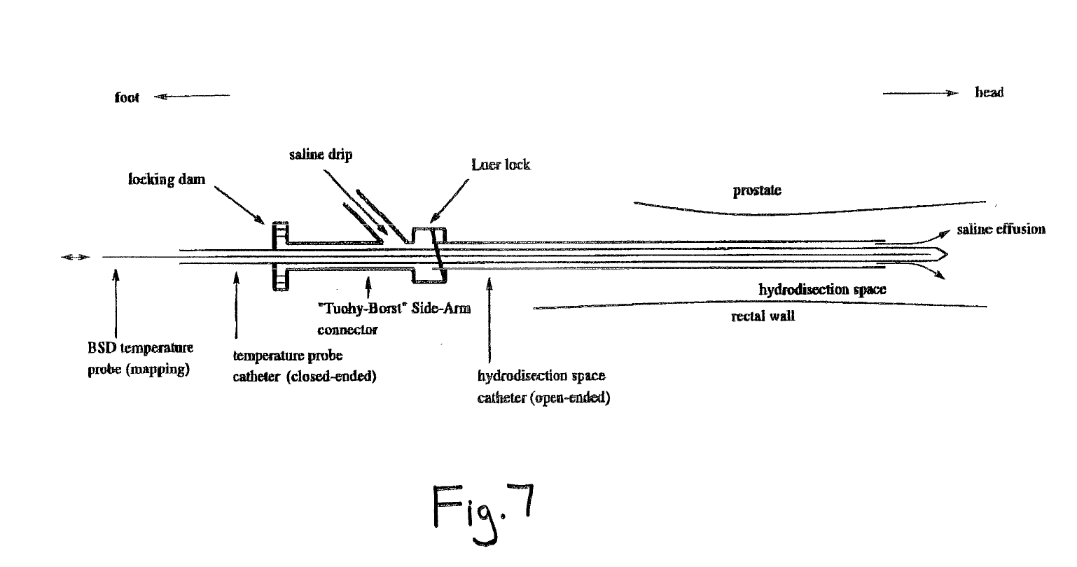

FIG. 7 shows a schematic of the new design for easy assembly of the hydrodisection space unit, and the convenient saline and temperature probe access through this unit. The outer conduit is a modified Flexi-Needle (supplied by Best Medical International, VA). The Flexi-Needle comes as a closed-ended, pointed teflon catheter (13 Ga., approx. 8 French, 15 cm length) Luer-locked to a square-ended insertion trocar. This two-piece set was also designed for radioisotope delivery. This setwas modified in our laboratory by removing the trocar, grinding the insertion end of the trocar to a sharp point with an electric grinder, cutting the pointed, closed-ended tip of the catheter, and replacing the trocar. This permits easy implantation of the unit due to the sharp trocar, and leaves an open end inside the hydrodisection space once the trocar is removed, through which room temperature saline may flow.

As observed in FIG. 7, a “Tuohy-Borst” Side-Arm connector (Cook Canada Inc.) Luer locks onto the hydrodisection space conduit. The temperature probe catheter is passed straight through the locking dam of the connector, into the hydrodisection space. The locking dam consists of a thumb-wheel surrounding a rubber seal. As the wheel is turned, the seal tightens against the catheter to secure it. Then, the temperature probe is fed through the catheter. The saline supply Luer-locks to the angled side-arm portion of the connector. As observed in the FIG. 7, saline flows freely through the connector and around the temperature probe catheter into the hydrodisection space.

In addition, new temperature probe catheters (16 Ga, 13″ length) supplied by Best Medical International are used for the BSD temperature probes in the hydrodisection space and rectal cooling units. These were purchased to prevent the need to open packages of splittable needle sets only to use the enclosed temperature probe catheter while discarding the splittable need portion of the set.

Throughout this application, various publications are referenced by author and year. Full citations for the publications are listed below. The disclosures of these publications in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### REFERENCES

Anscher M. S., Dewhirst M. W., Prosnitz, L. R., Dodge R., Samulski, T. V., Combined external beam irradiation and external regional hyperthermia for locally advanced adenocarcinoma of the prostate. Int. J. Radiat. Oncol. Biol. Phys:, 1997: 15; 37(5); 1059-65.

Gelet A., Dubernard J. M., Cathignol D, Blanc E., Souchon R., Pangaud C., Bouvier R., Chapelon J. Y., Preliminary results of the treatment of 44 patients with localized cancer of the prostate using transrectal focused ultrasound. Prog. Urol., 1998: 8(1); 68-77.

Gelet A., Dubernard J. M., Cathignol D, Abdelrahim, A. F., Souchon R., Pangaud C., Bouvier R., Chapelon J. Y., Treatment of prostate cancer with transrectal focused ultrasound: early clinical experience. Prog. Urol, 1996: 29(2); 174-83.

Mendecki J., Rriedenthal E., Botstein C., Paglione R., Sterzer F., Microwave applicators for localized hyperthermia treatment of cancer of the prostate. Int. J. Rad. Oncol. Biol. Phys., 1980: 6; 1583-1588.

Yershalmi A., Servadio C., Leib Z., Fishelovitz Y., Stein J. A., Localized hyperthermia for treatment of carcinoma of the prostate: a preliminary report. Prostate, 1982: 3; 623-630.

What is claimed is:

1. A hydrodissection apparatus, for treating the prostate, comprising:
moving means for moving the prostate away from adjacent rectum; and
treatment means for treating the prostate, and protecting the rectum from the treatment.

2. A hydrodissection apparatus, for treating the prostate, comprising:
moving means for moving the prostate away from adjacent rectum; and
treatment means for treating the prostate, with the rectum being protected from the treatment.

3. The hydrodissection apparatus of claim 2, wherein said moving means further including a fluid flow means for moving the prostate and cooling adjacent structures.

4. The hydrodissection apparatus of claim 3, wherein said fluid flow means further includes a needle and sheath assembly in fluid communication with a fluid.

5. The hydrodissection apparatus of claim 4, wherein said fluid is a continuous saline solution drip infusion.

6. The hydrodissection apparatus of claim 4, wherein said fluid flow means further comprises a urethral cooling assembly.

7. The hydrodissection apparatus of claim 6, wherein said urethral cooling asssembly further comprises an anchor balloon operably attached to said urethral cooling assembly.

8. The hydrodissection apparatus of claim 7, wherein said treatment means further includes;
a transrectal ultrasound transducer means for monitoring temperature and hydrodissection;
a microwave power source electrically connected to said transrectal ultrasound transducer means for creating microwave energy;
a microwave antenna assembly means electrically connected to said microwave power source for distribution of said microwave energy;
at least two sensor thermosensor arrays operatively connected to said transrectal ultrasound transducer means for detecting and monitoring tissue temperature;
a needle and sheath assembly means operatively connected to said microwave power source for placement of said microwave antenna assembly means and said two sensor thermosensor array; and
a rectal probe containing at least two thermosensors operatively connected to said transrectal ultrasound transducer means for monitoring rectal temperature.

9. A method of treating the prostate of a patient, utilizing a hydrodissection apparatus, comprising the steps of:
moving the prostate away from adjacent rectum; and
treating the prostate, with the rectum being protected from the treatment.

10. The method of claim 9, wherein a hydrodissection space is created by inserting a needle and sheath assembly into said recto-prostatic space and further connecting said needle and sheath assembly to a fluid flow.

11. The method of claim 10, wherein said fluid flow is further defined as a continuous saline solution drip.

12. The method of claim 11, wherein a single array thermosensor is inserted via a Y-connector through a needle and sheath assembly.

13. The method of claim 12, wherein said fluid flow of said moving step further includes a urethral cooling assembly.

14. The method of claim 13, wherein said urethral cooling assembly further includes an anchor balloon operably attached to said urethral cooling assembly and is inserted inside the urinary bladder.

15. The method of claim 14, wherein said anchor balloon is inflated with 7 cc of sterile water and traction is applied to ensure proper positioning.

16. The method of claim 15, wherein said fluid flow further includes a temperature sensing device for monitoring the temperature within said hydrodissection space.

17. The method of claim 16, wherein said temperature sensing device adjusts said fluid flow based upon said temperature within said hydrodissection space.

18. The method of claim 17, wherein when said temperature sensing device receives a reading of greater than 45 degrees Celsius said flow rate is increased.

19. The method of claim 18, further including a power controlling device which shuts off power when said fluid flow does not decrease said temperature.

20. The method of claim 19, wherein said power controlling device shuts down in 2.5 watt increments.

21. A method of providing thermal therapy to prostate tissue of a patient, comprising the steps of:
providing a fluid flow to a location adjacent a portion of the patient's prostate and the patient's rectum 14, said location selected to allow said fluid flow to begin physically separating the portion of the prostate and the rectum;
said fluid flow causing physical separation of the portion of the prostate and the rectum and applying the thermal therapy to the prostate tissue.

22. The method as defined in claim 21, further including the step of continuing to provide said fluid flow to substantially completely physically separate the prostate and the rectum with said fluid flow.

23. The method as defined in claim 21, wherein said location is disposed in a biplane fascial layer of the patient.

24. The method as defined in claim 21, wherein delivery of said fluid flow is pressurized.

25. The method as defined in claim 21, wherein said fluid flow is recirculated.

26. The method as defined in claim 21, wherein said temperature of said fluid flow is monitored with a temperature sensor disposed in said fluid flow.

27. The method as defined in claim 21, wherein said fluid flow cools the rectum.

28. The method as defined in claim 21, wherein said fluid flow is a liquid.

29. The method as defined in claim 21, wherein said fluid flow is a gel .

30. A method of providing high intensity thermal therapy to prostate tissue of a patient, comprising the steps of:
providing a thermal therapy delivery system including a therapeutic element and an exterior portion capable of being cooled;
physically separating a portion of the patient's prostate rectum using said thermal therapy delivery system;
providing energy to said therapeutic element, said therapy element directing more energy toward the prostate than the rectum to heat the prostate to temperature levels in excess of conventional hyperthermia treatment temperature levels; and
cooling said exterior portion of said thermal therapy delivery system at least at those locations where said exterior portion contacts patient tissue.

31. The method as defined in claim 20, wherein said exterior is cooled 360 degrees radially.

32. A hydrodissection apparatus, for treating tissue, comprising:
moving means for moving the treated tissue away from the adjacent tissue; and
treatment means for treating the treated tissue, and protecting the second mentioned tissue from the treatment.

33. A method of treating the tissue of a patient, utilizing a hydrodissection apparatus, comprising the steps of:
moving the treated tissue away from adjacent tissue; and
treating the treated tissue, with the second mentioned tissue being protected from the treatment.

34. A method for determining the expected thermal damage volume of a tissue, comprising the steps of:
placing antennas and probes in the tissue to be treated;
scanning and digitizing the information received from the antennas and probes;
calculating the heating pattern of the antennas using known heating patterns of antennas; and
producing a three-dimensional temperature map of the expected thermal damage volume as a function of time during the treatment.
